(19) United States
(12) Patent Application Publication
(10) Pub. No.: US 2011/0217288 A1
(43) Pub. Date: Sep. 8, 2011

(54) Involvement of Androgen/Androgen Receptor Pathway in Fabry Disease

(75) Inventors: Jinsong Shen, Plano, TX (US); Raphael Schiffmann, Rockwall, TX (US); Xingli Meng, Plano, TX (US)

(73) Assignee: Baylor Research Institute, Dallas, TX (US)

(21) Appl. No.: 13/038,293

(22) Filed: Mar. 1, 2011

Related U.S. Application Data
(60) Provisional application No. 61/310,802, filed on Mar. 5, 2010.

(51) Int. Cl. A61K 39/395; A61K 38/00; A61K 38/15; A61P 3/00; G01N 33/573; C12Q 1/68; A61B 17/00

(52) U.S. Cl. 424/130.1; 514/1.1; 435/7.4; 435/6.12; 606/135

(57) ABSTRACT

Novel therapies for the treatment of Fabry disease by using androgen/androgen receptor (AR) pathway-related molecules as biomarkers and use of approaches targeting androgen/AR pathway are presented herein. The involvement of aberrant androgen/AR pathway in Fabry disease has never been previously described. The present invention describes, (i) use of approaches that target androgen/AR pathway as therapeutic treatments for Fabry disease and (2) use of the levels of androgen/AR pathway-related molecules in body fluids or tissues as biomarkers for evaluation of disease progression and efficacy of treatments in Fabry patients.

### CROSS-REFERENCE TO RELATED APPLICATIONS

This application claims priority to U.S. Provisional Patent Application No. 61/310,802, filed Mar. 5, 2010, the entire contents of which are incorporated herein by reference.

### TECHNICAL FIELD OF THE INVENTION

The present invention relates in general to the field of Fabry disease biomarkers, and more particularly to the measurement of the levels of androgen/androgen-receptor pathway related molecules in body fluids or tissues as biomarkers for the evaluation of disease progression and efficacy of treatments in Fabry patients.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with the use of androgen/androgen receptor (AR) pathway molecules as biomarkers and with agents targeting androgen/AR pathway for treating various diseases.

United States Patent Application 20090282496 (Chang, 2009) discloses methods and compositions related to the inhibition of the interaction of androgen and androgen receptor for the treatment of bladder cancer. The Chang invention involves administering to a subject a vector comprising an agent that inhibits one or more activities of androgen or androgen receptor (AR) gene operably linked to a bladder specific promoter. The agent described in the application is an anti-androgen or anti-androgen receptor (AR) small interfering RNA (siRNA).

WIPO Publication No. WO/2009/111881 (Wiseman et al., 2009) discloses a method of diagnosing differentiated thyroid cancer in a subject is described. The method comprises obtaining a thyroid tissue sample from the subject; determining, in the sample, a level of each member of a plurality of biomarkers in the sample, and comparing the levels determined against a reference to determine if the levels indicate differentiated thyroid cancer. The plurality of biomarkers may be a panel comprising: (a) Galectin-3, P16 and androgen receptor; (b) Galectin-3, P16 and HBME-1; or (c) Cytokeratin 19 and Vascular Endothelial Growth Factor, each of which may be used in conjunction with additional biomarkers.

### SUMMARY OF THE INVENTION

The present invention describes a novel approach that targets the androgen/androgen receptor (AR) pathway as therapeutic treatments for Fabry disease. Further the present invention also describes the measurement of the levels of androgen/AR pathway-related molecules in body fluids or tissues as biomarkers for evaluation of disease progression and efficacy of treatments in Fabry patients.

The present invention in one embodiment discloses a method of diagnosing Fabry's disease in a subject comprising the steps of: collecting a sample from the subject suspected of having Fabry's disease, comparing the levels of one or more molecules selected from the group consisting of an androgen, an androgen precursor, an androgen metabolite, an androgen-receptor (AR) pathway molecule, and one or more AR target genes and related metabolites in the sample from the subject suspected of having Fabry's disease with that of a normal subject not having Fabry's disease, and determining whether the subject has Fabry's disease based on a statistically significant change in the levels of the one or more molecules in the samples between the subject suspected of having Fabry's disease and the normal subject. In one aspect of the method of the present invention the sample is a biological fluid sample. In another aspect the one or more molecules comprise testosterone, dihydrotestosterone (DHT) and precursors, congeners, salts, complexes, analogs of testosterone and DHT, insulin-like growth factor 1 (IGF-I), and Phosphorylated Akt. In yet another aspect the method comprises a comparison of a ratio between the Phosphorylated Akt to a total Akt in the samples between the subject suspected of having Fabry's disease and the normal subject. In one aspect, the ratio of the Phosphorylated Akt to total Akt is increased in the sample of the subject suspected of having Fabry's disease. In another aspect the subject suspected of having Fabry's disease may optionally show a hypertrophy of one or more organs selected from a heart, a kidney, a liver, and a spleen.

The instant invention in another embodiment provides for a method of monitoring the efficacy of a therapeutic intervention on a patient suffering from Fabry's disease comprising the steps of: (i) administering one or more pharmaceutical compositions to the patient at one or more pre-defined intervals, wherein the pharmaceutical composition comprises one or more therapeutic agents against Fabry's disease, (ii) collecting a sample from the patient, (iii) comparing the levels of one or more molecules selected from the group consisting of an androgen, an androgen precursor, an androgen metabolite, an androgen-receptor (AR) pathway molecule, and one or more AR target genes and related metabolites in the sample from the patient undergoing the therapeutic intervention with that of the same patient prior to the commencement of the therapeutic intervention and (iv) determining whether the therapeutic intervention is effective based on the comparison of the measured levels of the one of more molecules in the sample from the patient before and after the commencement of the therapeutic intervention. The sample used in the method disclosed herein is a biological fluid sample. In a related aspect the method of the present invention further comprises the step of continuing, terminating, or modifying the therapeutic intervention based on the comparison of the measured levels of the androgen, the androgen precursor, the androgen metabolite, the androgen-receptor (AR) pathway molecule, and one or more AR target genes and related metabolites before and after the commencement of the therapeutic intervention. The modification of the therapeutic intervention involves an increase or a decrease in a dosage, a frequency or both of the one or more pharmaceutical compositions or combinations thereof.

In one aspect of the method the one or more molecules comprise testosterone, dihydrotestosterone (DHT) and precursors, congeners, salts, complexes, analogs of testosterone and DHT, insulin-like growth factor 1 (IGF-I), and Phosphorylated Akt. In another aspect a decreased level of the IGF-I, the Phosphorylated Akt or both in the sample of the patient after the commencement of the therapeutic intervention is indicative of a successful therapeutic intervention. In a specific aspect the method comprises a measurement of a ratio between the Phosphorylated Akt to a total Akt in the sample of the patient before and after the commencement of the therapeutic intervention, wherein a decrease in the ratio of the Phosphorylated Akt to total Akt is indicative of the successful therapeutic intervention. In yet another aspect the therapeutic intervention comprises medical or surgical castration, androgen synthesis blockers, 5α-reductase enzyme inhibitors, AR gene expression knockdown agents, inhibitors of HSP90, AR pathway kinase blocking agents, enzyme replacement therapies, histone deacetylases inhibitors, pain medications, dialysis, organ transplantation, diet modifications or any combinations thereof.

Yet another embodiment of the present invention describes a pharmaceutical composition for treating Fabry's disease in a patient comprising: a therapeutically effective amount of at least one of an androgen synthesis blocker, a 5α-reductase enzyme inhibitors, an androgen-receptor (AR) gene expression knockdown agent, an inhibitor of HSP90, an AR pathway kinase blocking agents or a histone deacetylases inhibitors sufficient to treat Fabry's disease dissolved, dispersed or suspended in an aqueous or a non-aqueous solvent, one or more optional related co-factors, proteins, antibodies, pain medications and other pharmaceutically active agents dissolved, dispersed, or suspended in an aqueous or a non-aqueous solvent, and one or more optional excipients, fillers, diluents, extended or controlled release agents, bulking agents, antiadherents, binders, lubricants, preservatives or any combinations thereof.

In one aspect the pharmaceutical composition comprises antiandrogens, flutamide, cyproterone acetate, ketoconazole, spironolactone, finasteride, geldanamycin and related analogues and derivatives, valproic acid, suberoylanilide hydroxamic acid (SAHA), Romidespin, (2E)-N-hydroxy-3-[4-({[2-(2-methyl-1H-indol-3-yl)ethyl]amino}methyl)phenyl]acrylamide, N-(2-Aminophenyl)-4-[[(4-pyridin-3-ylpyrimidin-2-yl)amino]methyl]benzamide or any combinations thereof. In another aspect the pharmaceutical composition is infused, administered subcutaneously, intravenously, peritoneally, orally, and intramuscularly. In yet another aspect the composition decreases a level insulin-like growth factor 1 (IGF-I), and Phosphorylated Akt in a biological sample of the patient taking the pharmaceutical composition when compared to the sample of the patient not taking the pharmaceutical composition. In a specific aspect the composition decreases a ratio of the Phosphorylated Akt to total Akt in the biological sample of the patient taking the pharmaceutical composition when compared to the sample of the patient not taking the pharmaceutical composition.

The present invention in one embodiment provides for a method of treating Fabry's disease in one or more subjects comprising the steps of: identifying the one or more subjects in need of treatment against Fabry's disease and administering one or more pharmaceutical compositions comprising a therapeutically effective amount at least one of an androgen synthesis blocker, a 5α-reductase enzyme inhibitors, an androgen-receptor (AR) gene expression knockdown agent, an inhibitor of HSP90, an AR pathway kinase blocking agents or a histone deacetylases inhibitors sufficient to treat Fabry's disease. The method of treatment further comprises the steps of monitoring the progression of Fabry's disease following the administration of the pharmaceutical composition, wherein the monitoring comprises measuring a level of at least one of an insulin-like growth factor 1 (IGF-I), a Phosphorylated Akt, and a ratio of the Phosphorylated Akt to total Akt following treatment and comparing the measured levels with the measured levels prior to the treatment.

A related aspect of the method comprises the step of terminating, continuing or modifying the treatment based on the levels of the insulin-like growth factor 1 (IGF-I), the Phosphorylated Akt, and the ratio of the Phosphorylated Akt to total Akt before and after the commencement of the treatment, wherein the modification comprises an increase or a decrease in a dosage, a frequency or both of the one or more pharmaceutical compositions or combinations thereof. In a specific aspect the pharmaceutical composition comprises antiandrogens, flutamide, cyproterone acetate, ketoconazole, spironolactone, finasteride, geldanamycin and related analogues and derivatives, valproic acid, suberoylanilide hydroxamic acid (SAHA), Romidespin, (2E)-N-hydroxy-3-[4-({[2-(2-methyl-1H-indol-3-yl)ethyl]amino}methyl)phenyl]acrylamide, N-(2-Aminophenyl)-4-[[(4-pyridin-3-ylpyrimidin-2-yl)amino]methyl]benzamide or any combinations thereof. In yet another aspect the pharmaceutical composition is infused, administered subcutaneously, intravenously, peritoneally, orally, and intramuscularly.

In another aspect the instant invention discloses a method of treating Fabry's disease in one or more subjects comprising the steps of: identifying the one or more subjects in need of treatment against Fabry's disease and performing a surgical or medical castration on the subject in need of treatment against the Fabry's disease. The method further comprises the steps of monitoring the progression of Fabry's disease following the surgical or medical castration, wherein the monitoring comprises measuring a level of at least one of an insulin-like growth factor 1 (IGF-I), a Phosphorylated Akt, and a ratio of the Phosphorylated Akt to total Akt following the castration treatment and comparing the measured levels with the measured levels prior to the castration. In a specific aspect a decreased level of the IGF-I, the Phosphorylated Akt or both in the sample of the patient after the castration is indicative of a successful treatment. In a related aspect a decrease in the ratio of the Phosphorylated Akt to total Akt after the castration is indicative of the successful treatment.

### BRIEF DESCRIPTION OF THE DRAWINGS

For a more complete understanding of the features and advantages of the present invention, reference is now made to the detailed description of the invention along with the accompanying figures and in which:

FIG. 1 is a plot showing the expression of one of the AR-target genes, insulin-like growth factor 1 (IGF-1), is significantly increased in 19-month old Fabry male mouse heart compared to age-matched wild type male mice;

FIG. 2A shows that Phosphorylated Akt, the downstream molecule of IGF-1, was increased significantly in 19-month male Fabry mouse heart compared to controls. However, there were no significant changes of IGF-1 expression and Akt activation in 5-month old Fabry male mouse heart suggesting that these changes are correlated with disease progression and severity. The densities of the bands in FIG. 2A were analyzed by NIH Image and the ratio of phosphorylated Akt to total Akt was calculated. There was 5.8-fold increase of the ratio in 19-month old male Fabry mouse heart in comparison to the controls;

FIG. 2B is a plot of the ratio of phosphorylated- and total-Akt;

FIG. 3 is a plot showing the 4-fold upregulation in the expression level of another target gene of AR activation, KLK3 (Prostate-specific antigen, PSA) in the endothelial cells of a Fabry patient compared to control cells; and

FIGS. 4A-4D are plots showing hypertrophy occurring in heart (4A) and kidney (4B) but not liver (4C) and spleen (4D) in Fabry mouse. Organ weight to body weight (BW) ratio was significantly increased in heart and kidney in 12-month old Fabry mouse compared to age-, and sex-matched wild type controls.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein a “biomarker” is virtually any biological compound, such as a protein and a fragment thereof, a peptide, a polypeptide, a proteoglycan, a glycoprotein, a lipoprotein, a carbohydrate, a lipid, a nucleic acid, an organic on inorganic chemical, a natural polymer, and a small molecule, that is present in the biological sample and that may be isolated from, or measured in, the biological sample. Furthermore, a biomarker can be the entire intact molecule, or it can be a portion thereof that may be partially functional or recognized, for example, by an antibody or other specific binding protein. A biomarker is considered to be informative if a measurable aspect of the biomarker is associated with a given state of the patient, for example a particular stage of sepsis. Such a measurable aspect may include, for example, the presence, absence, or concentration of the biomarker in the biological sample from the individual and/or its presence as part of a profile of biomarkers.

As used herein, the term “androgen” refers to: testosterone; dihydrotestosterone (DHT); and precursors, congeners, salts, complexes, and analogs of testosterone and DHT. Examples of precursors of testosterone and DHT include, for example, DHEA, pregnenolone, progesterone, 17-OH-progesterone, and androsterone. Examples of analogs of testosterone and DHT include: testosterone esters, including straight and branched C 1-18 alkyl esters (herein referred to as “simple alkyl esters”), for example, testosterone enanthate, testosterone propionate, testosterone undecanoate, and testosterone heptylate, and cycloaliphatic esters, for example, testosterone cypionate, testosterone cyclopentyl alkyl ester, and testosterone cyclohexyl alkyl ester; and the analogous esters of DHT.

As used herein, the term “receptor” includes, for example, molecules that reside on the surface of cells and mediate activation of the cells by activating ligands, but also is used generically to mean any molecule that binds specifically to a counterpart. One member of a specific binding pair would arbitrarily be called a “receptor” and the other a “ligand”. No particular physiological function need be associated with this specific binding. Thus, for example, a “receptor” might include antibodies, immunologically reactive portions of antibodies, molecules that are designed to complement other molecules, and so forth. Indeed, in the context of the present invention, the distinction between “receptor” and “ligand” is entirely irrelevant; the invention concerns pairs of molecules which specifically bind each other with greater affinity than either binds other molecules. However, for ease of explanation, the invention method will be discussed in terms of target receptor (again, simply a molecule for which a counterpart is sought that will react or bind with it) and “ligand” simply represents that counterpart.

As used herein the term “androgen receptor” refers to a ligand-dependent transcription factor belonging to the nuclear steroid hormone receptor superfamily. The “androgen receptor” is the essential mediator for androgen action. Androgens can enhance androgen receptor protein levels by increasing the half-life, as well as by stimulating the phosphorylation of the androgen receptor. Phosphorylation may affect numerous characteristics of nuclear receptors including ligand binding, nuclear translocation, dimerization, DNA binding, and protein-protein interactions.

As used herein, the term “treatment” refers to the treatment of the conditions mentioned herein, particularly in a patient who demonstrates symptoms of the disease or disorder.

As used herein, the term “treatment” or “treating” refers to any administration of a compound of the present invention and includes (i) inhibiting the disease in an animal that is experiencing or displaying the pathology or symptomatology of the diseased (i.e., arresting further development of the pathology and/or symptomatology) or (ii) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

The terms “effective amount” or “therapeutically effective amount” described herein means the amount of the subject compound that will elicit the biological or medical response of a tissue, system, animal or human that is being sought by the researcher, veterinarian, medical doctor or other clinician.

The terms “administration of” or “administering a” compound as used herein should be understood to mean providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The term “pharmaceutically acceptable” as used herein to describe a carrier, diluent or excipient must be compatible with the other ingredients of the formulation and not deleterious to the recipient thereof.

As used herein, the term “Glycosphingolipids (GSLs) includes amphipathic compounds consisting of sugar and ceramide moieties, are ubiquitous components of the plasma membrane of all vertebrate cells.

The term “knock-outs” as used herein also include, e.g., conditional knock-outs, wherein alteration of the target gene can be activated by exposure of the animal to a substance that promotes target gene alteration, introduction of an enzyme that promotes recombination at the target gene site (e.g., Cre in the Cre-lox system), or other method for directing the target gene alteration.

The term “transfection” as used herein refers to the introduction of foreign DNA into eukaryotic cells. Transfection may be accomplished by a variety of means known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Thus, the term “stable transfection” or “stably transfected” refers to the introduction and integration of foreign DNA into the genome of the transfected cell. The term “stable transfectant” refers to a cell which has stably integrated foreign DNA into the genomic DNA. The term also encompasses cells which transiently express the inserted DNA or RNA for limited periods of time. Thus, the term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells which have taken up foreign DNA but have failed to integrate this DNA.

As used herein, the term “polymerase chain reaction” (PCR) refers to the method of K. B. Mullis U.S. Pat. Nos. 4,683,195, 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as DCTP or DATP, into the amplified segment). In addition to genomic DNA, any oligonucleotide sequence can be amplified with the appropriate set of primer molecules. In particular the amplified segments created by the PCR process itself are, themselves, efficient templates for subsequent PCR amplifications.

The present invention describes an approach involving the measurement of the levels of androgen/AR pathway-related molecules (e.g. androgen level and expression levels of AR and AR-target genes) in body fluids and tissues as biomarkers to determine severity of Fabry disease progression and also as biomarkers to evaluate metabolic correction of various therapies for Fabry disease.

Further, the invention also describes approaches that target androgen/AR pathway are useful therapeutic treatments for Fabry disease, including medical or surgical castration, agents blocking androgen synthesis, inhibitors of 5α-reductase (enzyme for converting testosterone to dihydrotestosterone), anti-androgens (androgen antagonists), knockdown of AR gene expression, inhibitors of HSP90 (a molecular chaperon in which prevents AR degradation), agents blocking any kinase in the pathways that can enhance AR signaling, inhibitors of co-activators that increase AR-mediated transcription, inhibitors of histone deacetylases which is required for optimal AR-mediated transcription.

Fabry disease is an inborn error of glycosphingolipid catabolism caused by an insufficient activity of α-galactosidase A and progressive accumulation of globotriaosylceramide and related glycosphingolipids in various tissues. Fabry disease is a multisystem disease. Major clinical manifestations include cardiac abnormalities (cardiac hypertrophy, arrhythmia, valvular disease, and myocardial infarction), progressive renal failure and vascular complications. At present, the mechanism of the disease is unknown. There is no good biomarker that can be used to estimate disease progression and to evaluate the efficacy of therapeutic treatments. The only available specific therapy for Fabry disease is enzyme replacement therapy (ERT). However, ERT has limited efficacy in many of clinical manifestations^{[1]}.

To elucidate the mechanism of Fabry disease and to develop new and better treatments, the present inventors studied the pathophysiology of the disease using the mouse model of Fabry disease (knockout mouse in which the gene encoding α-galactosidase A was made inoperative by homologous recombination) and patients' tissues and cell culture as well. Androgens and androgen receptor (AR) play critical roles in variety of physiological and pathological conditions. Besides sexual differentiation (e.g. sex characteristics of male and spermatogenesis), androgens generally promote protein synthesis and growth of the tissues expressing AR. It has been known that androgen causes hypertrophy of cardiomyocytes [2] and also increases kidney size [3]. Although research results for androgen effects on cardiovascular system are controversial so far, laboratory studies demonstrated that androgens play roles in development of some cardiovascular diseases such as atherosclerosis [4, 5] The major pathway of androgen action involves androgen binding to AR. Upon activation by androgens, AR transports from the cytosol into cell nucleus, binds to specific sequence of DNA known as androgen response element (ARE) in some genes (AR-target genes), and modulates transcription of these genes to control cell behavior. Androgens also exhibit other effects that are not mediated by transcription or protein synthesis (referred as non-genomic effects).

Animals: The colonies of Fabry mice [6] and control mice with the same genetic background were maintained in the laboratory of the inventors under standard housing condition. At the indicated time points, mice were sacrificed after measurement of body weight. Heart, Kidney, liver and spleen were dissected, weighed and flash frozen in liquid nitrogen and were stored in −80° C. until use.

Electrocardiography (ECG): Electrocardiograms were recorded non-invasively in conscious mice (without anesthesia or surgery) using ECGenie system (Mouse Specific Inc). Analyzed data include heart rate, heart rate variability, RR interval, PR interval, QRS interval and QT and QTc intervals.

Cell culture: Dermal microvascular endothelial cell line derived from Fabry patient (IMFE1, [7]) was cultured in EGM-2 medium (Lonza). To correct disease phenotypes, the cells were infected with retroviral vector expressing normal human α-galactosidase A. The transduced cells were selected by fluorescence-activated cell sorting (FACS) for Green Fluorescent Protein (GFP) which was expressed simultaneously with α-galactosidase A through internal ribosome entry site (IRES). As mock treatment, cells were infected with the vector without α-galactosidase A gene and were sorted as above.

Quantitative RT-PCR: RNA was extracted from heart and cultured cells using RNeasy Kit (Qiagen). Reverse transcription reaction was performed using SuperScript II (Invitrogen). Pre-designed TaqMan probe and primers for mouse IGF-1 was purchased from Applied Biosystems (ABI). Pre-designed primers for human KLK3 was purchased from SABiosciences. 18s rRNA was used as internal control and detected by TaqMan probe and primers (ABI).

Western blot analysis: Mouse hearts were homogenized in RIPA buffer with protease inhibitors (Santa Cruz Biotech). Protein concentrations were determined with BCA protein assay kit (Pierce). Electrophoresis and blotting were done as described [7]. The membrane was labeled with rabbit monoclonal antibody to phosphorylated Akt (Cell Signaling Tech). To detect total Akt, the membrane was stripped and re-probed with antibody to Akt (pan) (Cell Signaling Tech). The densities of the bands were analyzed by NIH Image and the ratio of phosphorylated Akt to total Akt was calculated

From the findings on Fabry mouse and cultured endothelial cells derived from Fabry patient, the present inventors hypothesize that androgen/AR pathway is abnormally activated in Fabry disease and that this abnormality plays a crucial role in major clinical manifestations of Fabry disease. However, in some instances, the pathway may be abnormally down-regulated. Evidence of upregulation includes:

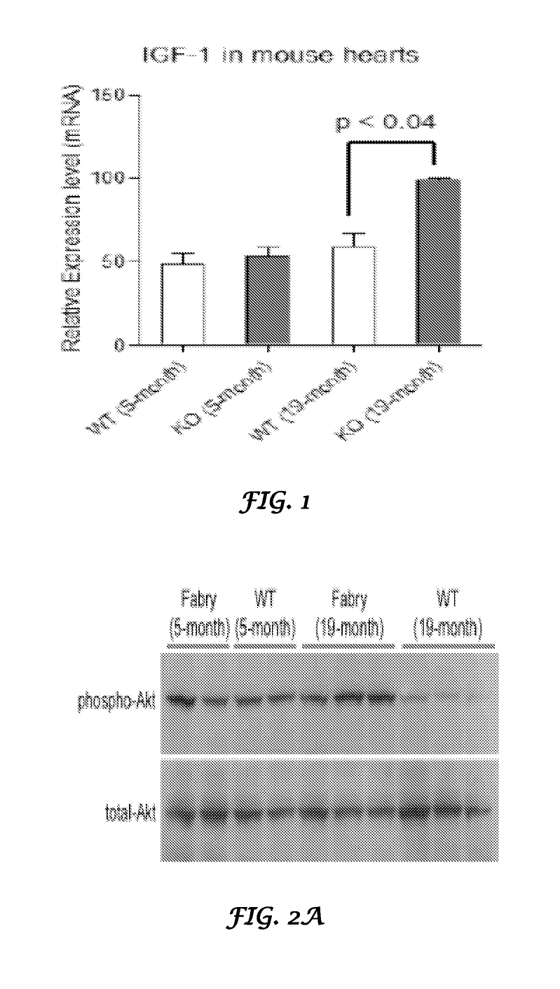

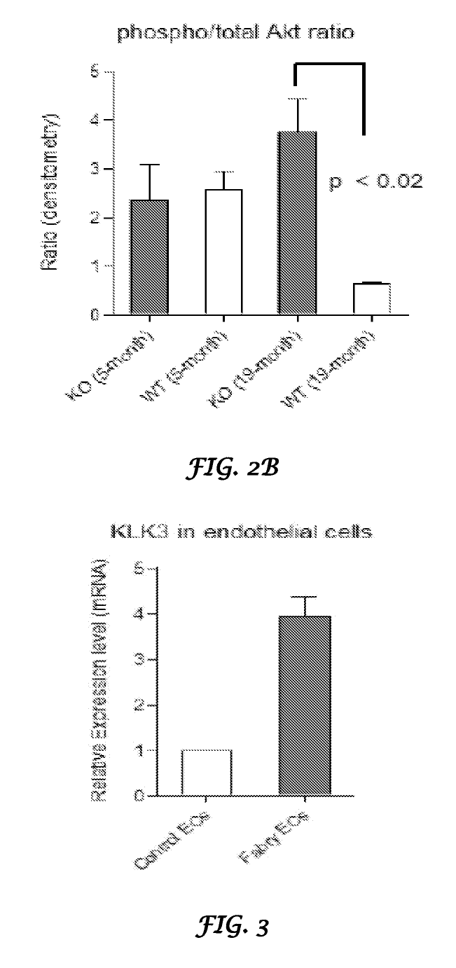

Abnormal activation of androgen/AR pathway in Fabry mouse heart: Fabry male mice show age-dependent cardiac hypertrophy and ECG abnormalities. The expression of one of the AR-target genes, insulin-like growth factor 1 (IGF-I), was significantly increased in 19-month old Fabry male mouse heart compared to age-matched wild type male mice (increased to 1.7-fold) (FIG. 1). Phosphorylated Akt, the downstream molecule of IGF-I, was increased significantly in these male Fabry mouse heart compared to controls (The ratio of phosphorylated- and total-Akt increased to 5.8-fold) (FIGS. 2A and 2B). However, there were no significant changes of IGF-1 expression and Akt activation in 5-month old Fabry male mouse heart suggesting that these changes are correlated with disease progression and severity. Fabry female mice also show cardiac hypertrophy and increased IGF-I expression in the heart compared to age-matched control female mice. However, Fabry female mice did not exhibit significant ECG abnormality.

Abnormal activation of androgen/AR pathway in cultured endothelial cells derived from Fabry patient: The expression level of another target gene of AR activation, KLK3 (Prostate-specific antigen, PSA) was upregulated to 4-fold in Fabry patient endothelial cells compared to control cells (the same cell line corrected by ectopic expression of normal α-galactosidase A) (FIG. 3).

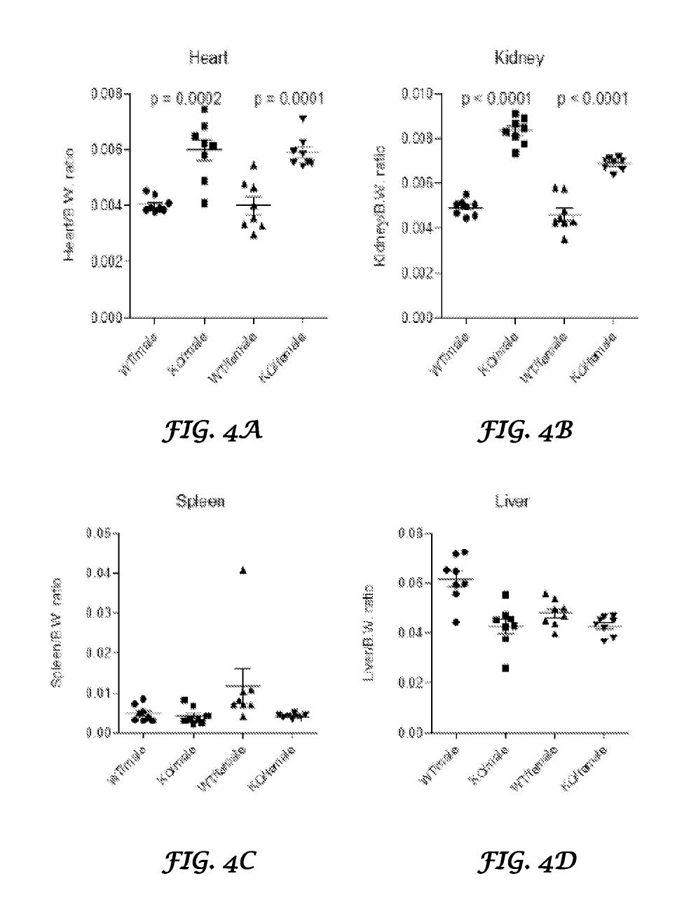

Other indirect evidence: Hypertrophy occurs in heart and kidney (FIGS. 4A and 4B) but not liver and spleen (FIGS. 4C and 4D) in Fabry mouse: Organ weight to body weight (BW) ratio was significantly increased in heart and kidney in 12-month old Fabry mouse compared to age-, and sex-matched wild type controls. There was no such increment in other organs such as liver and spleen. These data in combination with previous studies demonstrating androgen effect on cardiac and kidney hypertrophy [2,3] provide indirect evidence for increased AR pathway activation in Fabry mouse.

Similar, cardiac hypertrophy, arrhythmia and abnormal vasodilation in Fabry patients [1] can be explained by enhanced AR activation (the latter can be caused by nongenomic effects of androgen on endothelium-independent relaxation [8]). It is well known that sex hormones influence intra-cardiac repolarization, signal conduction and the expression of calcium channels [9, 10]. The inventors found significant electrocardiographic (EKG) abnormalities in male Fabry KO mice. Compared to age and genetic background matched controls, 11 months old male Fabry mice had statistically significantly lower heart rate, increased heart rate variability, longer PR and ST intervals. However, female homozygous KO mice of the same age, while having no alpha-galactosidase A activity like the males, had virtually no EKG abnormalities.

The present invention discloses that the levels of androgen/AR pathway-related molecules (e.g. androgen level and expression levels of AR and AR-target genes) in body fluids and tissues are: (1) biomarkers of severity of Fabry disease progression and also (2) biomarkers to evaluate metabolic correction of various therapies for Fabry disease. In addition, the inventors hypothesize that approaches that target androgen/AR pathway are useful therapeutic treatments for Fabry disease. These include medical or surgical castration, agents blocking androgen synthesis, inhibitors of 5α-reductase (enzyme for converting testosterone to dihydrotestosterone), anti-androgens (androgen antagonists), knockdown of AR gene expression, inhibitors of HSP90 (a molecular chaperonin which prevents AR degradation), agents blocking any kinase in the pathways that can enhance AR signaling, inhibitors of co-activators that increase AR-mediated transcription, inhibitors of histone deacetylases which is required for optimal AR-mediated transcription.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

United States Patent Application 20090282496: Androgen receptor related methods for treating bladder cancer.
WIPO Publication No. WO/2009/111881: Biomarkers for diagnosis of differentiated thyroid cancer.
1. Schiffmann R (2009) Fabry disease. Pharmacol Ther 122(1):65-77.
2. Marsh J O, et at. (1998) Androgen receptors mediate hypertrophy in cardiac myocytes. Circulation 98(3):256-261.
3. Shukla A, Shukla G S, & Radin N S (1992) Control of kidney size by sex hormones: possible involvement of glucosylceramide. Am J Physiol 262(1 Pt 2):F24-29.
4. McCrohon J A, Jessup W, Handelsman O J, & Celermajer O S (1999) Androgen exposure increases human monocyte adhesion to vascular endothelium and endothelial cell expression of vascular cell adhesion molecule-1. Circulation 99(17):2317-2322.
5. Fujimoto R, et al. (1994) Androgen receptors, 5 alpha-reductase activity and androgen dependent proliferation of vascular smooth muscle cells. J Steroid Biochem Mol Bioi 50(3-4): 169-174.
6. Ohshima T, et al (1997) α-Galactosidase A deficient mice: A model of Fabry disease. Proc. Natl. Acad. Sci. USA 94: 2540-2544.
7. Shen J. S, et al (2007) Establishment and characterization of Fabry disease endothelial cells with an extended lifespan. Mol. Genet. Metab 92: 137-144.
8. Yue P, Chatterjee K, Beale C, Poole-Wilson P A, & Collins P (1995) Testosterone relaxes rabbit coronary arteries and aorta. Circulation 91(4):1154-1160.
9. Fulop L, et al. (2006) Effects of sex hormones on ECG parameters and expression of cardiac ion channels in dogs. Acta Physiol (Oxf) 188(3-4):163-171.
10. Brouillette J, Rivard K, Lizotte E, & Fiset C (2005) Sex and strain differences in adult mouse cardiac repolarization: importance of androgens. Cardiovasc Res 65(1):148-157.

What is claimed is:

1. A method of diagnosing Fabry's disease in a subject comprising the steps of:
collecting a sample from the subject suspected of having Fabry's disease;
comparing a level of one or more molecules selected from the group consisting of an androgen, an androgen precursor, an androgen metabolite, an androgen-receptor (AR) pathway molecule, and one or more AR target genes and related metabolites in the sample from the subject suspected of having Fabry's disease with that of a normal subject not having Fabry's disease; and
determining whether the subject has Fabry's disease based on a statistically significant change in the levels of the one or more molecules in the samples between the subject suspected of having Fabry's disease and the normal subject.

2. The method of claim 1, wherein the sample is a biological fluid sample.

3. The method of claim 1, wherein the one or more molecules comprise testosterone, dihydrotestosterone (DHT) and precursors, congeners, salts, complexes, analogs of testosterone and DHT, insulin-like growth factor 1 (IGF-I), and Phosphorylated Akt.

4. The method of claim 1, wherein the method comprises a comparison of a ratio between the Phosphorylated Akt to a total Akt in the samples between the subject suspected of having Fabry's disease and the normal subject.

5. The method of claim 4, wherein the ratio of the Phosphorylated Akt to total Akt is increased in the sample of the subject suspected of having Fabry's disease.

6. The method of claim 1, wherein the subject suspected of having Fabry's disease may optionally show a hypertrophy of one or more organs selected from a heart, a kidney, a liver, and a spleen.

7. A method of monitoring the efficacy of a therapeutic intervention on a patient suffering from Fabry's disease comprising the steps of:
administering one or more pharmaceutical compositions to the patient at one or more pre-defined intervals, wherein the pharmaceutical composition comprises one or more therapeutic agents against Fabry's disease;
collecting a sample from the patient;
comparing a level of one or more molecules selected from the group consisting of an androgen, an androgen precursor, an androgen metabolite, an androgen-receptor (AR) pathway molecule, and one or more AR target genes and related metabolites in the sample from the patient undergoing the therapeutic intervention with that of the same patient prior to the commencement of the therapeutic intervention; and
determining whether the therapeutic intervention is effective based on the comparison of a measured level of the one of more molecules in the sample from the patient before and after the commencement of the therapeutic intervention.

8. The method of claim 7, wherein the sample is a biological fluid sample.

9. The method of claim 7, further comprising the step of terminating the therapeutic intervention based on the comparison of the measured levels of the androgen, the androgen precursor, the androgen metabolite, the androgen-receptor (AR) pathway molecule, and one or more AR target genes and related metabolites before and after the commencement of the therapeutic intervention.

10. The method of claim 7, further comprising the step of continuing the therapeutic intervention based on the comparison of the measured levels of the androgen, the androgen precursor, the androgen metabolite, the androgen-receptor (AR) pathway molecule, and AR target genes and related metabolites before and after the commencement of the therapeutic intervention.

11. The method of claim 7, further comprising the step of modifying the therapeutic intervention based on the comparison of the measured levels of the androgen, the androgen precursor, the androgen metabolite, the androgen-receptor (AR) pathway molecule, and AR target genes and related metabolites before and after the commencement of the therapeutic intervention, wherein the modification comprises an increase or a decrease in a dosage, a frequency or both of the one or more pharmaceutical compositions or combinations thereof.

12. The method of claim 7, wherein the one or more molecules comprise testosterone, dihydrotestosterone (DHT) and precursors, congeners, salts, complexes, analogs of testosterone and DHT, insulin-like growth factor 1 (IGF-I), and Phosphorylated Akt.

13. The method of claim 7, wherein a decreased level of the IGF-I, the Phosphorylated Akt or both in the sample of the patient after the commencement of the therapeutic intervention is indicative of a successful therapeutic intervention.

14. The method of claim 7, wherein the method comprises a measurement of a ratio between the Phosphorylated Akt to a total Akt in the sample of the patient before and after the commencement of the therapeutic intervention.

15. The method of claim 14, wherein a decrease in the ratio of the Phosphorylated Akt to total Akt is indicative of the successful therapeutic intervention.

16. The method of claim 7, wherein the therapeutic intervention comprises medical or surgical castration, androgen synthesis blockers, 5α-reductase enzyme inhibitors, AR gene expression knockdown agents, inhibitors of HSP90, AR pathway kinase blocking agents, enzyme replacement therapies, histone deacetylases inhibitors, pain medications, dialysis, organ transplantation, diet modifications or any combinations thereof.

17. A pharmaceutical composition for treating Fabry's disease in a patient comprising:
a therapeutically effective amount of at least one of an androgen synthesis blocker, a 5α-reductase enzyme inhibitors, an androgen-receptor (AR) gene expression knockdown agent, an inhibitor of HSP90, an AR pathway kinase blocking agents or a histone deacetylases inhibitors sufficient to treat Fabry's disease dissolved, dispersed or suspended in an aqueous or a non-aqueous solvent;
one or more optional related co-factors, proteins, antibodies, pain medications, and other pharmaceutically active agents dissolved, dispersed, or suspended in an aqueous or a non-aqueous solvent; and
one or more optional excipients, fillers, diluents, extended or controlled release agents, bulking agents, antiadherents, binders, lubricants, preservatives or any combinations thereof.

18. The composition of claim 17, wherein the pharmaceutical composition comprises antiandrogens, flutamide, cyproterone acetate, ketoconazole, spironolactone, finasteride, geldanamycin and related analogues and derivatives, valproic acid, suberoylanilide hydroxamic acid (SAHA), Romidespin, (2E)-N-hydroxy-3-[4-({[2-(2-methyl-1H-indol-3-yl)ethyl]amino}methyl)phenyl]acrylamide, N-(2-Aminophenyl)-4-[[(4-pyridin-3-ylpyrimidin-2-yl)amino]methyl]benzamide or any combinations thereof.

19. The composition of claim 17, wherein the pharmaceutical composition is infused, administered subcutaneously, intravenously, peritoneally, orally, and intramuscularly.

20. The composition of claim 17, wherein the composition decreases a level insulin-like growth factor 1 (IGF-I), and Phosphorylated Akt in a biological sample of the patient taking the pharmaceutical composition when compared to the sample of the patient not taking the pharmaceutical composition.

21. The composition of claim 17, wherein the composition decreases a ratio of the Phosphorylated Akt to total Akt in the biological sample of the patient taking the pharmaceutical composition when compared to the sample of the patient not taking the pharmaceutical composition.

22. A method of treating Fabry's disease in one or more subjects comprising the steps of:
identifying the one or more subjects in need of treatment against Fabry's disease; and
administering one or more pharmaceutical compositions comprising a therapeutically effective amount at least one of an androgen synthesis blocker, a 5α-reductase enzyme inhibitors, an androgen-receptor (AR) gene expression knockdown agent, an inhibitor of HSP90, an AR pathway kinase blocking agents or a histone deacetylases inhibitors sufficient to treat Fabry's disease.

23. The method of claim 22, further comprising the steps of monitoring the progression of Fabry's disease following the administration of the pharmaceutical composition, wherein the monitoring comprises measuring a level of at least one of an insulin-like growth factor 1 (IGF-I), a Phosphorylated Akt, and a ratio of the Phosphorylated Akt to total Akt following treatment and comparing the measured levels with the measured levels prior to the treatment.

24. The method of claim 22, further comprising the step of terminating, continuing or modifying the treatment based on the levels of the insulin-like growth factor 1 (IGF-I), the Phosphorylated Akt, and the ratio of the Phosphorylated Akt to total Akt before and after the commencement of the treatment, wherein the modification comprises an increase or a decrease in a dosage, a frequency or both of the one or more pharmaceutical compositions or combinations thereof.

25. The method of claim 22, wherein the pharmaceutical composition comprises antiandrogens, flutamide, cyproterone acetate, ketoconazole, spironolactone, finasteride, geldanamycin and related analogues and derivatives, valproic acid, suberoylanilide hydroxamic acid (SAHA), Romidespin, (2E)-N-hydroxy-3-[4-({[2-(2-methyl-1H-indol-3-yl)ethyl]amino}methyl)phenyl]acrylamide, N-(2-Aminophenyl)-4-[[(4-pyridin-3-ylpyrimidin-2-yl)amino]methyl]benzamide or any combinations thereof.

26. The method of claim 22, wherein the pharmaceutical composition is infused, administered subcutaneously, intravenously, peritoneally, orally, and intramuscularly.

27. A method of treating Fabry's disease in one or more subjects comprising the steps of:
identifying the one or more subjects in need of treatment against Fabry's disease; and
performing a surgical or a medical castration on the subject in need of treatment against the Fabry's disease.

28. The method of claim 27, further comprising the steps of monitoring the progression of Fabry's disease following the surgical or medical castration, wherein the monitoring comprises measuring a level of at least one of an insulin-like growth factor 1 (IGF-I), a Phosphorylated Akt, and a ratio of the Phosphorylated Akt to total Akt following the castration treatment and comparing the measured levels with the measured levels prior to the castration.

29. The method of claim 27, wherein a decreased level of the IGF-I, the Phosphorylated Akt or both in the sample of the patient after the castration is indicative of a successful treatment.

30. The method of claim 27, wherein a decrease in the ratio of the Phosphorylated Akt to total Akt after the castration is indicative of the successful treatment.
